(19) United States
(12) Patent Application Publication
(10) Pub. No.: US 2014/0046181 A1
(43) Pub. Date: Feb. 13, 2014

(54) ACOUSTICALLY RESPONSIVE PARTICLES WITH DECREASED CAVITATION THRESHOLD

(71) Applicants: Michael Benchimol, San Diego, CA (US); Sadik C. Esener, Solana Beach, CA (US); Dmitri Simberg, San Diego, CA (US); Stuart Ibsen, La Jolla, CA (US)

(72) Inventors: Michael Benchimol, San Diego, CA (US); Sadik C. Esener, Solana Beach, CA (US); Dmitri Simberg, San Diego, CA (US); Stuart Ibsen, La Jolla, CA (US)

(73) Assignee: THE REGENTS OF THE UNIVERSITY OF CALIFORNIA, Oakland, CA (US)

(21) Appl. No.: 13/978,140

(22) PCT Filed: Jan. 5, 2012
(86) PCT No.: PCT/US2012/020389

§ 371 Date: Oct. 30, 2013

Related U.S. Application Data
(60) Provisional application No. 61/430,073, filed on Jan. 5, 2011.

(51) Int. Cl. A61K 41/00; A61M 5/00; A61M 37/00

(52) U.S. Cl. CPC A61K 41/0047; A61M 37/0092; A61M 5/007
USPC 600/431; 604/22; 424/490; 424/450; 424/94.1; 514/44 R; 424/9.5

(57) ABSTRACT

Techniques, systems, devices and materials are disclosed for implementing and fabricating drug delivery and imaging vehicles, which are activated in the body at a tissue of interest by focused ultrasound. In one aspect, a drug delivery vehicle can include a carrier having an outer membrane that envelopes an acoustic sensitizer particle and a payload substance to be delivered to the target tissue. The outer membrane can protect the acoustic sensitizer particle and the payload substance from degradation and opsonization. The outer membrane can be functionalized with a tumor targeting ligand to cause the drug delivery vehicle to selectively accumulate in a tumor region over other tissues, as well as with an agent to increase circulation time by reducing uptake from undesired body tissues, organs, and systems.

### CROSS-REFERENCE TO RELATED APPLICATIONS

This patent document claims priority of U.S. Provisional Patent Application No. 61/430,073, filed Jan. 5, 2011, entitled “ECHOGENIC PARTICLES WITH DECREASED CAVITATION THRESHOLD”; the entire disclosure of which is incorporated herein by reference for all purposes.

### STATEMENT REGARDING FEDERALLY SPONSORED RESEARCH OR DEVELOPMENT

This invention was made with government support under grant CA119335 awarded by the National Institutes of Health (NIH). The government has certain rights in the invention.

### BACKGROUND

This patent document relates to systems, devices, and processes related to ultrasound imaging and therapy technologies.

Ultrasound refers to sound waves operating at frequencies higher than that of the upper level of typical human hearing. Ultrasound signals can be used in a variety of biomedical and other applications for imaging and therapeutic purposes. For example, ultrasound imaging (also referred to as sonography) is a medical imaging modality that employs the properties of sound waves traveling through a medium to render a visual image of internal structures and functions of animals and humans. Ultrasound imaging can include contrast enhanced ultrasound, which utilizes a contrast medium to enhance an ultrasound image. For example, ultrasound contrast agents can reflect the ultrasound waves in a variety of ways from interfaces between the agents and this ability of reflecting the ultrasound waves of such agents is measured by the degree of echogenicity. Ultrasound contrast agents can include gas-filled micro-sized bubbles (microbubbles) that have a greater degree of echogenicity with respect to the surrounding tissue. For example, microbubbles can be used as ultrasound contrast agents to enhance the reflection of the ultrasound waves and produce a higher resolution image due to the high echogenicity difference. However, microbubble ultrasound contrast agents can have short in vivo circulation times, poor tissue extravasation, and short-lived ultrasound signal contrast enhancement due to their instability, e.g., rapid dissolution or coalescence resulting in larger microbubbles that provide little to no signal enhancement in standard contrast-sensitive modes of diagnostic ultrasound imaging systems.

Therapeutic applications of ultrasound can include focused ultrasound. Focused ultrasound can provide a safe, non-invasive means to deposit energy deep within the body with millimeter precision without causing adverse biological effects. For example, focused ultrasound can be used as a mechanism to release therapeutic compounds carried by a larger structure or particle (referred to as a ‘vehicle’) for targeted and controlled delivery to a particular region or tissue of the body. Examples of ultrasound delivery vehicles have included fluorocarbon-based microbubbles, in which the size of the microbubbles can be on the order of micrometers or more. However, the fluorocarbon-based microbubbles can be restricted to remain in the vasculature, and therefore their payloads must be delivered in the vasculature. Also, microbubbles can be unstable passing through the heart, lungs, and spleen, leading to a short circulation half-life.

### SUMMARY

Techniques, systems, and devices are described for implementing and fabricating drug delivery and imaging vehicles that can be activated in a particular location by acoustic energy.

In one aspect of the disclosed technology, an ultrasound system for delivering a substance loaded in an acoustically responsive particle includes a mechanism that supplies one or more particle each having an outer shell that encloses an aqueous medium containing an ultrasound-responsive nanoparticle and a payload substance and a mechanism that produces ultrasonic acoustic energy and focuses the ultrasonic acoustic energy at a particular region where the one or more particles are located to cause the ultrasound-responsive nanoparticle to rupture the outer shell of each of the one or more particles, thus releasing the payload substance within the particular region.

In another aspect, a drug delivery vehicle includes a carrier comprising an outer membrane that envelopes an acoustic sensitizer particle and a payload substance to be delivered in a body to a target tissue, in which the outer membrane protects the acoustic sensitizer particle and the payload substance from degradation and opsonization, an outer surface of the outer membrane is functionalized with a tumor targeting ligand to cause the drug delivery vehicle to selectively accumulate in a tumor region over other tissues, and the outer surface of the outer membrane is further functionalized with an agent to increase circulation time by reducing uptake from undesired body tissues, organs, and systems.

In another aspect, a method of delivering an ultrasound activated carrier to a target cell includes loading an ultrasound-responsive carrier with a payload comprising a cavitation nanoparticle to selectively deliver the payload to a target cell in a body through vasculature of the body, wherein the carrier extravasates from the vasculature to the target cell based on an enhanced permeability and retention effect; and applying a focused ultrasound pulse to the carrier to rupture the carrier and release the payload to the target cell.

In another aspect, a method for producing a stabilized microbubble includes applying a focused ultrasound pulse to a liposome containing a nanoemulsion structure in an aqueous medium to fragment the liposome, causing lipids of the fragmented liposome to rearrange into one or more microbubbles.

In another aspect, an ultrasound contrast agent device includes an outer membrane structured to form an enclosed chamber, an aqueous medium enclosed within the chamber, and one or more nanoemulsion structures in the aqueous medium within the chamber, in which the outer membrane is formed of a material that can be fragmented by a focused ultrasound pulse into components that rearrange into one or more microbubbles that are stabilized against coalescence and dissolution and operate as a contrast agent in ultrasound imaging.

In another aspect, an ultrasound responsive device for carrying a payload substance includes an outer membrane structured to form an enclosed chamber, an aqueous medium enclosed within the chamber, one or more ultrasound-responsive nanoparticles in the aqueous medium and structured to reduce an ultrasound cavitation threshold at an interface between each ultrasound-responsive nanoparticle and aqueous medium, and a payload substance in the aqueous medium, in which the outer membrane is formed of a material that can be fragmented by cavitation within the chamber under a focused ultrasound pulse to release the payload substance outside the enclosed chamber.

The subject matter described in this patent document can be implemented in specific ways to provide one or more of the following features. The disclosed technology can enable the delivery of payloads at a desired time directly to a desired location in the body with high specificity. For example, payloads can include drugs, imaging agents, enzymes, nucleic acids such as DNA and RNA, viral vectors, agents to facilitate the cell internalization of these payloads, or any other therapeutic or sensing particle or molecule. The disclosed technology can include an ultrasound-sensitive vehicle or carrier composed of liquid or solid particles that can encounter physiological pressures without being significantly affected. For example, the described vehicles can provide stability in ultrasound imaging and therapeutic implementations. The disclosed technology can include vehicles that can circulate like liposomes (e.g., which can pass through the heart and lungs many times) allowing a greater percentage of the vehicles to enter the tumor. Also, these exemplary vehicles can be small enough to aggregate in tumors by enhanced permeability and retention (EPR) and be taken up by biological cells. In some examples, the described techniques can be used to place ultrasound-triggered delivery or contrast agents inside the tumor or biological cells. Also, the disclosed technology can allow use of lower energy ultrasound, which can be safer to a subject. Additionally, the disclosed technology can be used as an imaging agent. For example, focused ultrasound can be used to convert liquid perfluorocarbon (PFC) gas nanodroplets into stabilized gas microbubbles for use in ultrasound contrast imaging. Also, the disclosed technology can include fabrication methods which provide many advantages and capabilities. For example, the disclosed technology can include an easy fabrication process capable of: large-scale manufacturing of liposomes; control of liposome size for use in a variety of applications; efficient incorporation of drugs, proteins, enzymes, biomolecules, nucleic acids, nanoparticles, microparticles, agents to facilitate cell internalization, and other solutes; incorporation of particles and emulsions for ultrasound-triggered payload release; incorporation of imaging/contrast agents; and creation of an artificial cell or a vesicle that can have cellular components capable of performing biological processes.

### BRIEF DESCRIPTION OF THE DRAWINGS

FIG. 1 shows a schematic of an exemplary ultrasound-responsive vehicle for payload delivery.

FIG. 2A shows an exemplary illustration of drug delivery using an ultrasound-responsive liposome.

FIG. 2B shows an exemplary illustration of transfection reagent delivery using an ultrasound-responsive liposome.

FIG. 2C shows an exemplary illustration of enzyme-prodrug application using an ultrasound-responsive liposome.

FIG. 3 shows an exemplary illustration of collagenase delivery by an ultrasound-responsive liposome.

FIGS. 4A and 4B show exemplary experimental data of the disclosed technology in gene expression.

FIG. 5 shows an exemplary schematic of acoustically-controlled production of a stabilized microbubble.

FIG. 6 shows exemplary images featuring fabrication of liposomes.

FIG. 7 shows an exemplary image sequence featuring destruction of liposomes by focused ultrasound in real time.

FIG. 8A shows an exemplary plot of encapsulation efficiency of IgG payload loading.

FIG. 8B shows an exemplary image of doxorubicin loaded into a liposome.

FIG. 9 shows another exemplary image sequence of liposome destruction by focused ultrasound.

FIG. 10 shows an exemplary plot representing quantification of % release of calcein.

FIG. 11 shows an exemplary plot of normalized enzyme activity using the disclosed technology.

FIG. 12 shows exemplary images of localized payload delivery in vivo.

FIG. 13 shows exemplary high speed images of a perfluorocarbon emulsion nucleating acoustic cavitation.

FIG. 14 shows exemplary images featuring cavitation threshold reduction observed with iron oxide nanoparticles under various experimental arrangements.

FIG. 15 shows an exemplary image of iron oxide nanoparticles one month after fabrication.

FIG. 16 shows an exemplary cryo-TEM micrograph of a liposome containing densely-packed perfluorononane emulsions.

Like reference symbols and designations in the various drawings indicate like elements.

### DETAILED DESCRIPTION

Techniques, systems, devices, and structures are described for implementing and fabricating drug delivery and imaging vehicles that can be activated in a particular location by acoustic energy.

Several embodiments of the drug delivery and imaging vehicles are described. In one aspect, the disclosed technology can include an acoustically responsive, echogenic particle or vehicle that can carry one or more substances (referred to as a “payload”) with a rupture mechanism that derives from one or more nanoparticles that can be activated by acoustic energy, e.g., focused ultrasound. The acoustically responsive vehicle can have an aqueous internal volume in which the payload and the nanoparticle can be contained. The nanoparticle can act as a nucleation site for acoustic cavitation thereby sensitizing the vehicle to acoustic (e.g., ultrasound) energy. The ultrasound trigger can result in rapid release of the vehicle's payload by destruction of the vehicle membrane. For example, acoustic energy focused (e.g., three dimensional (3D) focused ultrasound) at a target in the body in which the vehicle has been employed. Inside the focal region, the acoustic energy can trigger the rupturing of the acoustically responsive vehicle. Outside of the focal region, the energy concentration quickly diminishes to the point where there is no triggering of the rupture mechanism. For example, the 3D focused ultrasound can include a higher intensity, pulse energy, frequency and/or duty cycle than an exemplary interrogation ultrasound pulse, e.g., used in ultrasound imaging modalities. Implementation of the disclosed acoustically responsive vehicles can enable the delivery of payloads (e.g., drugs, imaging agents, enzymes, nucleic acids, viral vectors, or any other therapeutic or sensing particle or molecule) at a desired time directly to a desired location in the body with high specificity. This can be accomplished, for example, by external, focused ultrasound triggering to release the payload.

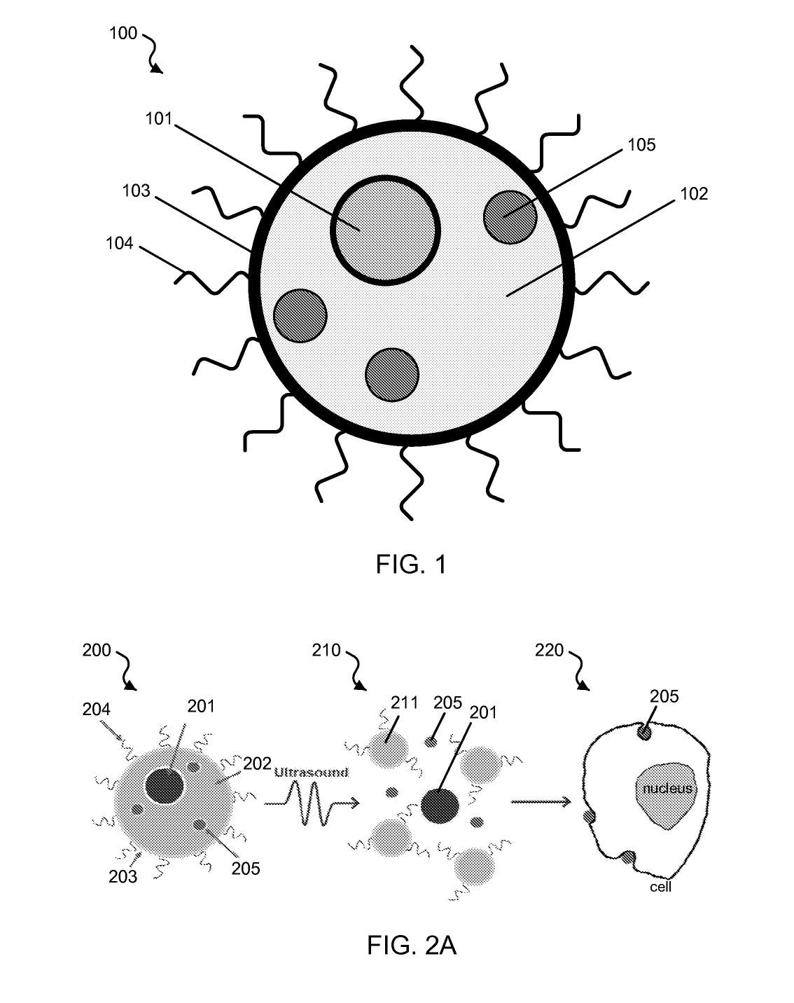

FIG. 1 shows an exemplary vehicle 100 that can include a nanoparticle 101 in an aqueous medium 102 enclosed by an outer shell 103 that can include externally protruding molecules 104. Vehicle 100 can also include payload substances 105. In this example, nanoparticle 101 and payload substances 105 are freely contained within medium 102. In another example, one or more nanoparticles and payload substances 105 can be freely contained within medium 102. In other examples, payload substances 105 can be contained within medium 102 on the inner boundary of outer shell 103 or on the outer boundary of outer shell 103. Examples of payload substances 105 can include drugs, imaging agents (e.g., iron oxide, gadolinium, radiotracers, fluorophores, etc.), enzymes, nucleic acids, viral vectors, or any other therapeutic or sensing particle or molecule.

Vehicle 100 can be configured as a liposome-based carrier with an outer shell 103 of a lipid bilayer. Additionally, vehicle 100 can be configured as a polymerosome-based carrier with an outer shell 103 of a polymer material. Vehicle 100 can also be configured as a biological cell. As shown in FIG. 1, the exemplary payload substances 105 are encapsulated in the internal volume of the vehicle 100 (e.g., medium 102) and therefore hidden from the host immune system during implementation.

Nanoparticle 101 can act as a nucleation site for acoustic cavitation, which is a violent process capable of destroying the vehicle membrane (e.g., outer shell 103) and resulting in rapid release of its payload, such as payload substances 105. Examples of nanoparticle 101 can include a liquid emulsion, metal, oxide, polymer, biomolecule, a solid particle stabilizing nanoscale pockets of gas, or a combination thereof. The presence of nanoparticle 101 can lower the threshold of acoustic energy (e.g., the intensity, pulse energy, and/or duty cycle of ultrasound) needed to initiate cavitation. For example, acoustic energy can be focused at a particular target in the body in which vehicle 100 is employed to trigger cavitation within a focused region of the acoustic energy; outside of the focal region, triggering of cavitation does not occur. For example, acoustic energy (e.g., focused ultrasound) can induce nanoparticle 101 to nucleate cavitation by a surface effect, or in examples of nanoparticle 101 being a nanoemulsion, by formation of a transient or stable bubble from a liquid emulsion. An exemplary surface effect can involve a gas cavity initiated at locations on the surface of the nanoparticle 101 induced by ultrasound pressure, which can be due to surface interactions of water molecules at the surface, or similar affects due to the hydrophobicity of the surface or the surface topology. An exemplary transient formation of a bubble from a nanoemulsion using ultrasound can involve the entrapped liquids in the nanoemulsion undergoing cavitation within the volume. The exemplary nucleated cavitation can produce a vapor bubble, which itself can subsequently serve as a nucleation site for further cavitation.

The exemplary outer shell 103 can include protruding molecules 104 that can be used, for example, to functionalize the vehicle 100 with a tumor targeting ligand to cause the delivery vehicles to preferentially accumulate in the tumor region (instead of other, non-tumor tissues). The exemplary outer shell 103 can also be functionalized with protruding molecules 104 that include polyethylene glycol (PEG) or other biomolecular agents, for example, to increase circulation time by reducing uptake, e.g., from undesired cells, tissues, organs or systems such as the immune system and the liver. Other biomolecular agents can include zwitterionic compounds or patient-specific coatings such as cell membranes. The materials of the outer shell 103 and protruding molecules 104 used to construct the exemplary drug delivery vehicle (e.g., vehicle 100) can be bioresorbable and non-accumulative within the body.

The disclosed acoustically responsive vehicles can be configured to have high encapsulation and flexibility of payloads. Exemplary acoustically responsive vehicles can be used for ultrasound contrast imaging and payload delivery under significantly different pharmacodynamics conditions. The disclosed acoustically responsive structures can exhibit significant stability in the body. For example, the disclosed acoustically responsive particles can be configured to be stable at high or low pressure environments, and are not easily destroyed, for example, in the heart and lungs.

For example, an acoustically responsive vehicle can be configured as an engineered liposome with an outer liposome membrane that envelopes the acoustic sensitizer, e.g., one or more nanoparticles, and a nano-sized payload that is protected from degradation and opsonization. The exemplary liposomes can be delivered to a specific target in the body for targeted release of the payload, e.g., as a drug delivery vehicle. This can be accomplished, for example, by external, focused ultrasound triggering to release the drug payload directly to the tumor site.

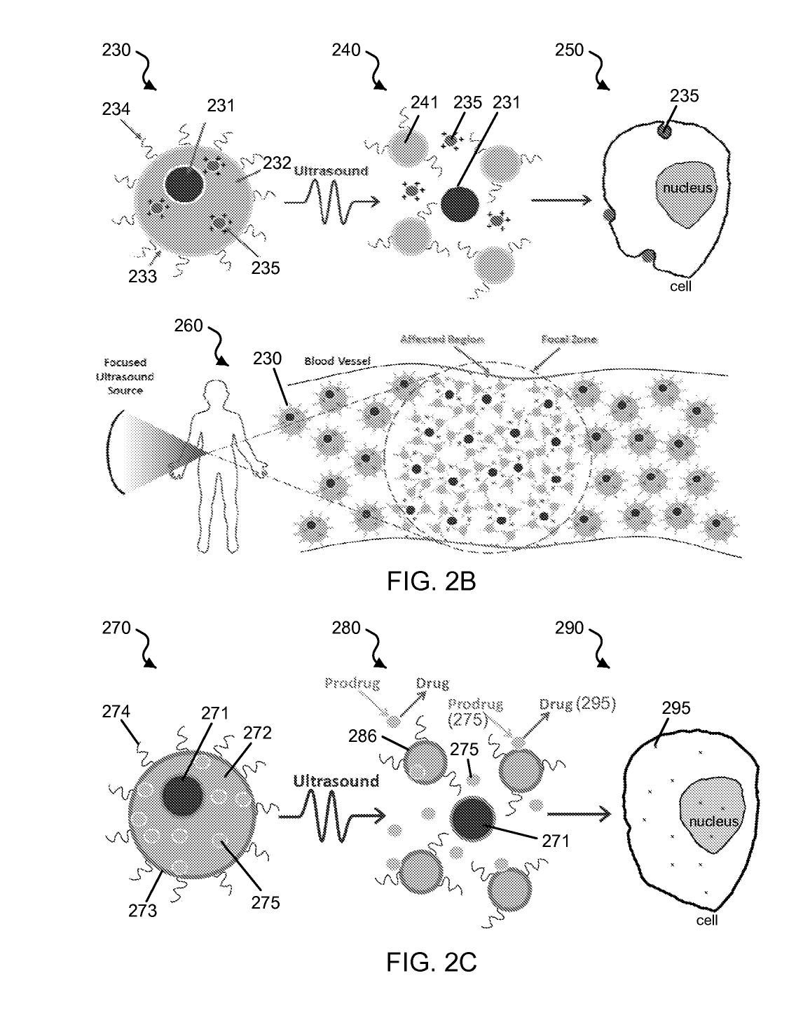

FIG. 2A shows a series of exemplary illustrations of a drug payload delivery using the engineered liposome-based ultrasound-responsive vehicle for a drug delivery application, e.g., chemotherapeutic drug delivery to a targeted tumor. An exemplary liposome vehicle 200 is shown that can include an ultrasound-sensitizing nanoparticle 201 in an aqueous medium 202 enclosed by an outer shell lipid bilayer 203 that can include molecular agents 204. Liposome vehicle 200 can include a payload substance of a drug 205. For example, drug 205 can include an anti-cancer chemotherapy drug or other non-cancer type drug. Molecular agents 204 can include molecules, compounds, or substances that can target a particular cell, tissue, organ, or structure in the body and/or increase circulation time, e.g., by reducing uptake from undesired body tissues, organs, and systems. Illustration 210 exemplifies liposome vehicle 200 after ultrasound has been applied, which can rupture the vehicles 200 and result in liposome fragments that can self-assemble to form new liposomes components 211 and release of the drug 205 that were initially encapsulated in the liposome vehicle 200. Illustration 220 shows cellular uptake of drug 205, e.g. by endocytosis.

In another example, acoustically responsive engineered liposomes with cavitation nanoparticles and nucleic acid payloads can be implemented for gene therapy to selected cells. For exemplary applications that include delivering a nucleic acid with the disclosed vehicle, it may be beneficial to also incorporate a transfection reagent, molecule, or particle that can improve the transfection efficiency. Encapsulation of a transfection reagent (TR) inside a vehicle (e.g., an engineered liposome) could extend its in vivo circulation time and subsequently be exposed to a targeted cell by implementation of the vehicle system with focused ultrasound (e.g., as shown in FIG. 2B).

FIG. 2B shows a series of exemplary illustrations of a plasmid-TR complex payload delivery using the engineered liposome-based ultrasound-responsive vehicle for a gene therapy application. An exemplary liposome vehicle 230 is shown that can include an ultrasound-sensitizing nanoparticle 231 in an aqueous medium 232 enclosed by an outer shell lipid bilayer 233 that can include PEG molecules 234. Liposome vehicle 230 can include a payload substance of a transfection reagent (plasmid-TR) complex 235. For example, plasmid-TR complex 205 can include a plasmid that is encapsulated with a cationic transfection reagent. Molecular agents 234 can include molecules, compounds, or substances that can target a particular cell, tissue, organ, or structure in the body and/or increase circulation time, e.g., by reducing uptake from undesired body tissues, organs, and systems. Illustration 240 exemplifies liposome vehicle 230 after ultrasound has been applied, which can rupture the vehicles 230 and result in liposome fragments that can self-assemble to form new liposomes components 241 and release of the plasmid-TR complex 235 that were initially encapsulated in the liposome vehicle 230. Illustration 250 shows cellular uptake of plasmid-TR complex 235, e.g. by endocytosis. In this example, ultrasound can be applied to rupture the vehicles 230 in a focused region (focal zone) as they circulate through the blood vessels using a focused ultrasound source, as shown in illustration 260 of FIG. 2B.

FIG. 2C shows a series of exemplary illustrations of a prodrug payload delivery using the engineered liposome-based ultrasound-responsive vehicle for a drug therapy application. An exemplary liposome vehicle 270 as shown can include cavitation nanoparticles 271 in an aqueous medium 272 enclosed by an outer shell lipid bilayer 273. The outer shell lipid bilayer 273 can include molecular agents 274. Molecular agents 274 can include molecules, compounds, or substances such as a targeting ligand (e.g., cyclic RGD), as well as molecules, compounds, or substances that increase circulation time, e.g., by reducing uptake from undesired body tissues, organs, and systems. Liposome vehicle 270 can include a payload substance of enzyme 275. For example, enzyme 275 can include a prodrug, which is a drug precursor molecule that can be converted into a more toxic form by some physical or chemical means. Illustration 280 exemplifies liposome vehicle 270 after ultrasound has been applied, which can rupture the vehicles 270 and result in liposome fragments that can self-assemble to form new liposomes components 286 and release of the prodrug-enzyme 275 that were initially encapsulated in the liposome vehicle 270. Illustration 280 also shows a conversion of enzyme (prodrug) 275 into a drug 295, which can be further taken up by cells, e.g. by endocytosis, as shown in illustration 290. In some examples, UV-blue light can be used to perform local photochemistry to turn a prodrug into its therapeutic form. Or, for example, other physical or chemical means can be applied to aid in the conversion prodrug-enzyme 275 to drug 295.

Dense extracellular collagen stroma can limit the penetration of drugs to tumor cells, e.g., pancreatic tumor cells. In another example, ultrasound-responsive engineered liposomes with cavitation nucleating nanoparticles and an enzymatic payload (e.g., collagenase) can be implemented to selectively deliver the payload to tumor tissue and increase permeability of the tumor to chemotherapy. The engineered liposomes can be injected intravenously and allowed to circulate throughout the body. The injected liposomes can extravasate (from vasculature to the target, e.g., tumor) due to the enhanced permeability and retention (EPR) effect. After extravasation, a focused ultrasound pulse can destroy the liposomes. Alternatively, ultrasound can be applied to burst the vehicles as they circulate through the vasculature of the tissue of interest. The burst-released payload can act on the desired target, e.g., the tumor, microenvironment or cancer cells. For example, the released collagenase payload can act to locally digest the extracellular tumor matrix, thereby allowing diffusion of subsequently administered chemotherapeutic drugs. It is noted that any collagenase that can leak into the systemic circulation can be inhibited by serum alpha-2-macroglobulin.

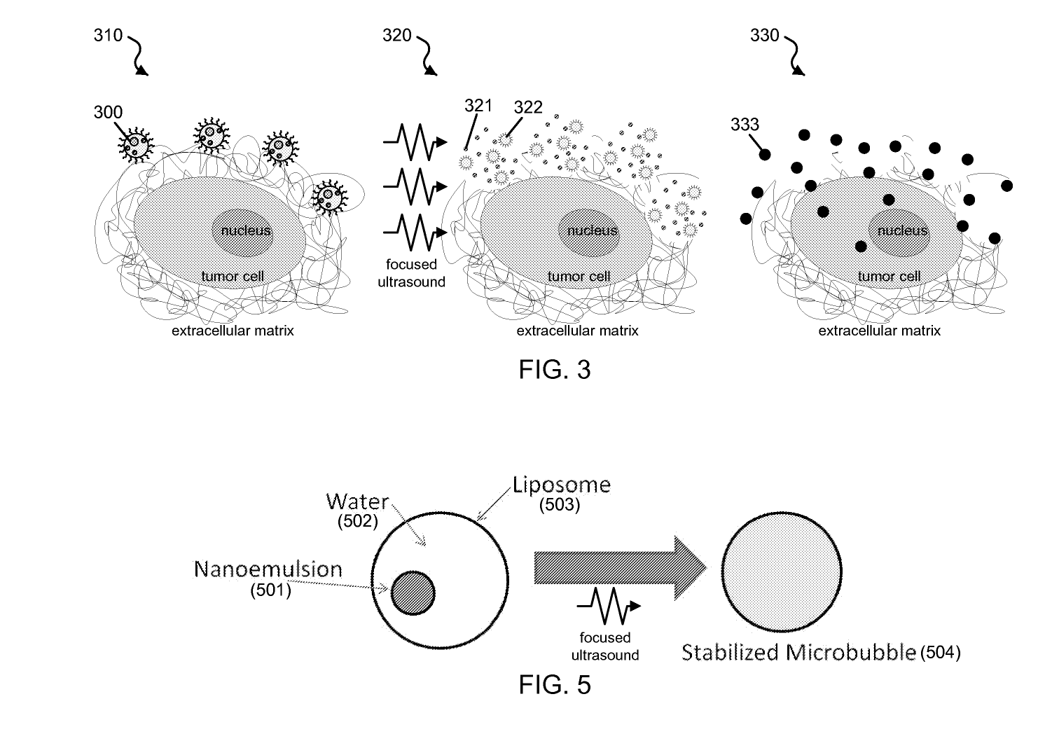

FIG. 3 shows a series of exemplary illustrations of a collagenase payload delivery using the engineered liposome-based ultrasound-responsive vehicle 300 to increase permeability of a tumor cell to chemotherapy drugs 333. Illustration 310 shows exemplary liposome vehicles 300 extravasated to a target tumor cell having an extracellular matrix. Liposome vehicle 300 can be configured to be ultrasound-sensitive liposomes with encapsulated ultrasound-sensitizing nanoparticles and a collagenase payload that can be ruptured by focused acoustic energy. The engineered liposomes can include targeting ligands on the liposome surface to pre-concentrate them in the vicinity of the targeted tumor. For example, the targeting ligands can include ligands of alpha v integrins, e.g., cyclic arginine-glycine-aspartic acid (RGD) peptide; which can target nano- and micro-structures to tumors and their vasculature. Illustration 320 shows a focused ultrasound pulse that bursts the liposome vehicles 300 to release the collagenase payload 321 (and remaining liposome fragments 322), promoting the digestion of tumor extracellular matrix. Illustration 330 shows subsequent access of exemplary chemotherapy drugs 333 and/or nanoparticles to the tumor cells. Repeated ultrasound treatments can be performed to further release fresh collagenase from newly accumulating liposomes to gradually digest the extracellular matrix.

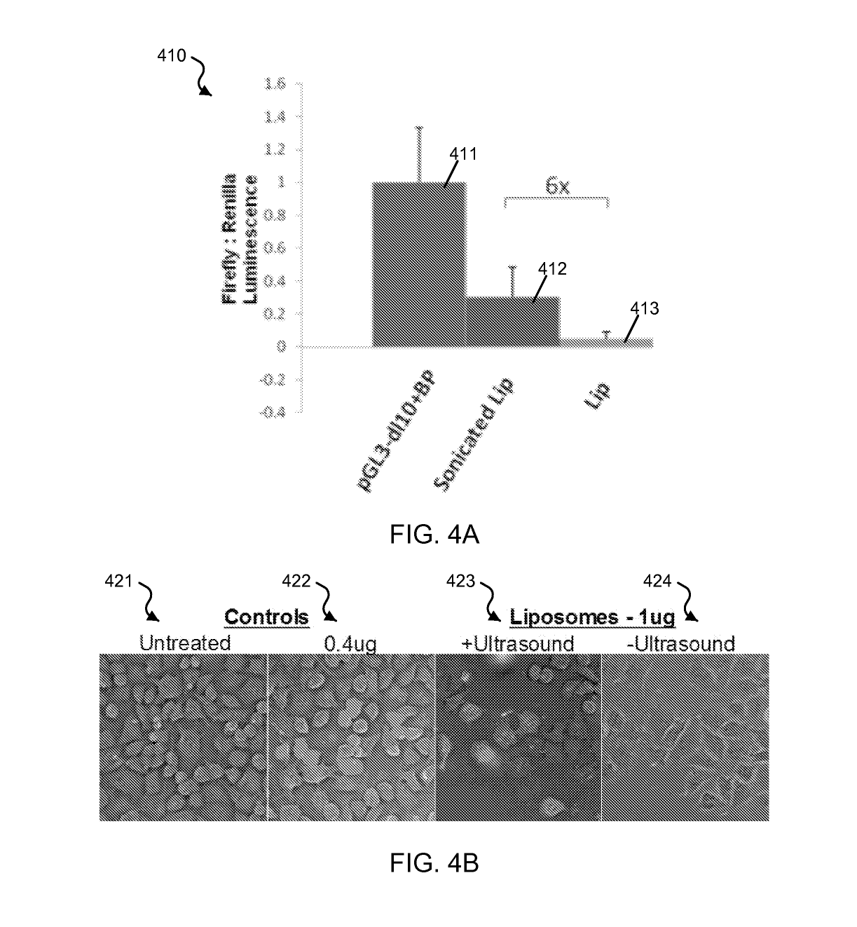

FIGS. 4A and 4B show exemplary experimental data of liposomal encapsulation of plasmid DNA (pDNA) and transfection reagent and ultrasound release resulting in increased gene expression. FIG. 4A shows an exemplary plot 410 that features firefly luciferase vector luminescence. In this exemplary experiment, firefly luciferase vector was encapsulated in the disclosed liposomes with a transfection reagent. The same concentration was used in the positive control (blue bar 411). A dual luciferase assay was used to normalize for other possible effects. Liposomes burst with ultrasound (red bar 412) showed a 6× increase in expression when compared to intact liposomes (green bar 413). FIG. 4B shows exemplary images featuring an experimental implementation of the disclosed technology for a gene expression application. In this example, cells were exposed or unexposed to a transfection reagent (e.g., eGFP vector) in the described conditions. Image 421 shows exemplary cells untreated by the eGFP vector containing liposomes. Image 422 shows exemplary cells treated by the eGFP vector without the use of the disclosed ultrasound-responsive vehicle (e.g., vehicle 100). Image 423 shows exemplary cells treated by the eGFP vector by implementing the disclosed technology, e.g. applying focused ultrasound to the cells exposed to the exemplary ultrasound-responsive liposomes encapsulating the eGFP vector. Image 424 shows exemplary cells treated by the ultrasound-responsive liposomes encapsulating the eGFP vector by not applying ultrasound, thereby maintaining liposomes encapsulating the eGFP vector relatively intact, and preventing subsequent transfection. Release of the pDNA with ultrasound resulted in greater expression, and more cells fluorescing.

In another example, the disclosed technology can be implemented to amplify a therapeutic effect by delivering a payload that can multiply at the target site, e.g., bursting ultrasound-responsive vehicles carrying a virus payload to a tumor site. For example, in the body, viruses can be taken up rapidly by the immune system and typically do not reach a tumor unaided. If viruses with the potential to destroy such tumor cells were able to reach the tumor and be taken up by the tumor cells, they potentially could kill the tumor cells by multiplying and causing a tumor-localized viral infection. The described technology can enable such a system, e.g., since the described acoustically responsive payload-delivery vehicle can protect the virus until it reaches the tumor bed, at which point it can be selectively released. For example, vehicle 100 (as shown in FIG. 1) can include payload substances 105 that are of viral vectors (e.g., programmed to attack specific tumor cells). Additionally, due to the stability of the disclosed acoustically responsive vehicles, e.g., to low and high pressures, pH, temperature, chemical substances, mechanical forces, and other environmental factors, the payload can be protected from the body's environment outside the focal region, as well as the healthy tissue of the body can be protected from the virus payload outside the focal region during transfer. For example, to promote targeted tumor specificity, the exemplary vehicle 100 can be configured with targeting ligands (e.g., cyclic RGD) as protruding molecules 104 on the outer shell 103.

In another aspect, the disclosed technology can include the conversion of nanodroplets or nanoemulsions to stabilized microbubbles for enhanced ultrasound imaging. Techniques are described to fabricate phase-changing emulsion structures to create stabilized gas microbubbles. For example, the disclosed technology includes exemplary processes for producing liposomes that can be used as cell-like compartments for the containment of molecules, nano-sized particles and micron-sized particles, e.g., such as biological machinery, imaging agents, and a variety of other payloads.

Although existing gas microbubble ultrasound contrast agents can provide contrast enhancement, they generally have short in vivo circulation times, poor tissue extravasation, and short-lived ultrasound signal contrast enhancement due to their instability. By implementing phase-shifting nanoemulsions of the described technology, these challenges and shortcomings of existing microbubble technologies can be circumvented. The exemplary phase-shifting nanoemulsions can produce microbubbles that are smaller, more stable, and have longer circulation in vivo. For example, the phase-changing emulsion can be packaged with the necessary lipids to produce a stabilized microbubble. Upon the application of the correct intensity and frequency of ultrasound, the exemplary nanodroplet or nanoemulsion encapsulated in a liposome can be converted into a microbubble, effectively creating the contrast agent onsite.

For example, a perfluorocarbon nanoemulsion can be encapsulated inside of a liposome to produce a stabilized microbubble for use as an ultrasound contrast agent. For example, lipids present in the liposome, once fragmented, can restructure around a newly formed gas microbubble and act as a stabilizer against microbubble coalescence and dissolution.

FIG. 5 shows an exemplary schematic of acoustically-controlled production of a stabilized microbubble. As shown in this example, nanoemulsion 501 can be encapsulated by liposome 503 in an aqueous medium such as water 502 to be converted into a microbubble in the presence of ultrasound, e.g., focused ultrasound. By applying focused ultrasound, lipids from the fragmented liposome can rearrange to form a stabilized microbubble 504.

The described carrier can be configured as a liposome, polymerosome, an inorganic or organic shell or capsid or even a biological cell including stem cell, macrophage or dentritic cells. As a liposome, the carrier can be made small (e.g., <200 nm) and can therefore be actively endocytosed by biological cells. For example, the small size and robust nature of the described carrier can lead to longer circulation times and tumor penetration by EPR.

The exemplary stabilized microbubble 504 can be configured as an ultrasound contrast agent. For example, stabilized microbubble 504 is an emulsion-based ultrasound contrast agent with properties that include a phase-shifting contrast agent packaged inside of a sub-micron liposome, which can contain necessary lipids to stabilize a newly-formed microbubble. The aqueous volume in between the emulsion and liposome can allow for emulsion expansion without much change in the acoustic properties. The lipids which comprise the liposome are free molecules and can become thermodynamically unstable as the force from the nanoemulsion expansion rips them from the liposome. These amphiphilic lipids can quickly stabilize themselves, for example, at the interface with an aqueous liquid and a hydrophobic gas. The choice of lipids for the liposome 503 can be optimized for microbubble stability. For example, since surfactants are generally not effective stabilizers for both gas microbubbles and liquid emulsion droplets, the disclosed technology may include independent control over the two shell materials.

As an example, implementation of the disclosed technology using an engineered liposome is described. For example, the engineered liposome can be fabricated by an exemplary process for making liposomes with a high incorporation of molecules, nanoparticles, or even micron-sized particles. For example, a particular solvent system can be implemented that can allow the formation of sheets of lipids. The solubility of lipids in this exemplary mixture can enable them to be stable in this lamellar state, without closing to form liposomes. Upon the addition of water, the lipid sheets can become thermodynamically unstable, which can cause them to close on themselves and form liposomes. Molecules or particles in the vicinity can be entrapped in these liposomes. Because of this specific state, the lipids can be well suspended around the incorporant before their formation. The exemplary liposomal delivery vehicle described in this example can use small (e.g., <200 nm) nanoparticles as an ultrasound sensitizer. This vehicle can be liquid or solid or a liquid or solid containing pockets of gas, and can have long-term stability and in vivo circulation time and can be actively taken up by cells through endocytosis. The particle itself need not undergo any chemical modification or phase change, and thus does not have to be a low-boiling point emulsion. The use of nanoparticles to decrease the required intensity/energy of ultrasound can allow the activation of the exemplary vehicles and reduction of bioeffects like heating, mechanical disruption, and nonspecific cavitation.

The exemplary stabilized microbubble 504 can be configured as a payload delivery vehicle that can carry a large aqueous payload, e.g., which can be rapidly released (e.g., <2 ms), as opposed to an oil payload space, which is limited by membrane diffusion, and can at best be fragmented into smaller droplets. For example, a payload loaded in or on the shell of a stabilized microbubble can similarly be still contained within particles after destruction. In contrast, the described system can contain a dissolved small molecule in the aqueous space. Once released, it can diffuse down its concentration gradient directly into the cytoplasm of a nearby cell instead of requiring endocytosis. This would overcome the associated challenges of degradation and endosomal escape.

The disclosed technology can include an exemplary fabrication technique to produce the described ultrasound-responsive liposomes using emulsification of perfluorocarbon (PFC) in a mixture of glycerol, propylene glycol and ethanol in the presence of lipids and emulsifying agents, and subsequent phase inversion. For example, the lipids can be dissolved in a mixture of propylene glycol, ethanol and glycerol. The percent of ethanol can be reduced to a minimum (e.g., <15%). The lipophilic dye 3,3′-dioctadecyloxacarbocyanine perchlorate (DiO) can be added to the ethanol solution to visualize the lipid bilayer, e.g., with a fluorescent microscope. This exemplary procedure can reproducibly generate liposomes of 1-10 μm diameter with particles encapsulated inside. The efficiency of nanoparticle and payload incorporation and overall shape can be associated with the lipid and solvent concentration used in the preparation.

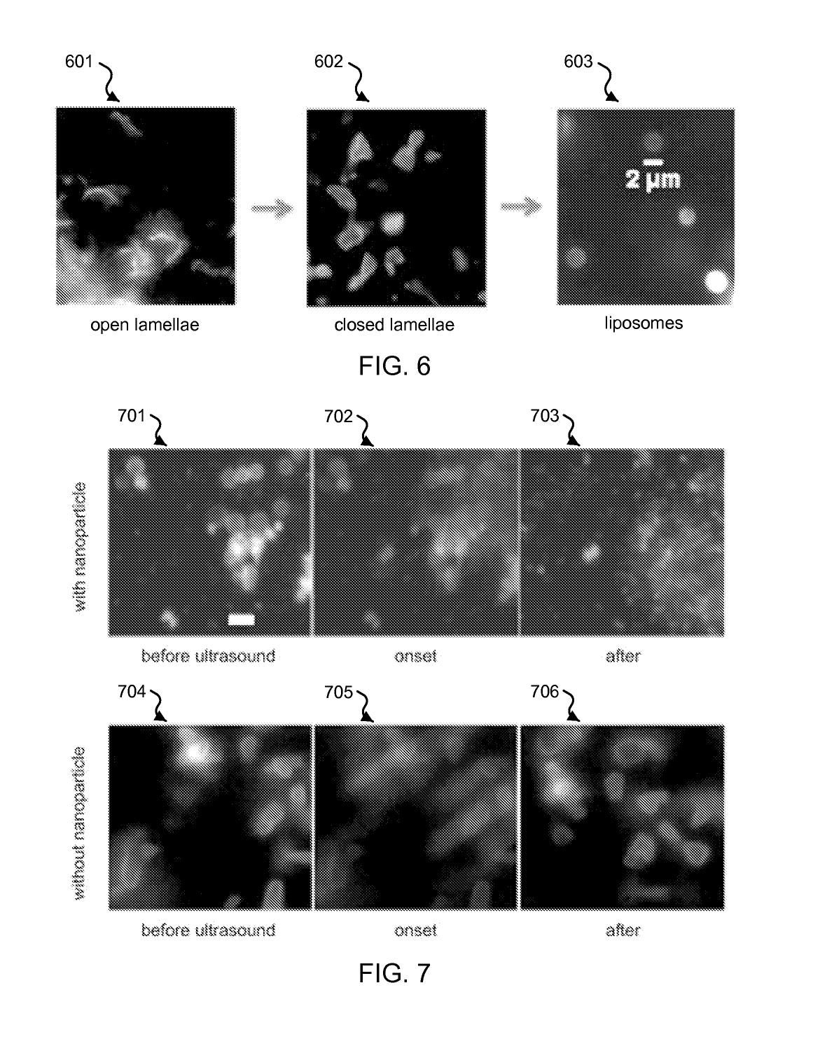

FIG. 6 shows exemplary images of a liposome manufacturing method. For example, image 601 shows open lamellae lipids, and image 602 shows closed lamellae lipids, after lipids were dissolved using an exemplary ethanol/glycerol/propylene glycol mixture. Liposomes can then form upon an increase of aqueous solvent content, as seen in image 603. In these exemplary images, lipid membranes were labeled with DiO. It is noted that the scale bar represents 2 μm, and the imaged region is not the same for all the images in FIG. 6.

An alternate fabrication method of the disclosed technology to produce the desired liposomes can include a reverse-phase evaporation (REV) process. In this process, emulsion droplets of water can be dispersed into an immiscible organic solvent. For example, diethyl ether, diisopropyl ether, chloroform, dichloromethane, and many others can be used. The aqueous volume can contain the payload to be entrapped. The organic solvent can contain the lipid that can make the liposome bilayer, and which also can serve initially to stabilize the nano-sized emulsion. Upon evaporation of the organic solvent, a gel phase can form, e.g., which is a large aggregate of water emulsions. Upon further evaporation and mixing, the gel can disperse into a liposome solution with sizes that can be determined by the initial emulsion size.

The interaction of dye-loaded liposomes and ultrasound was studied using a custom-built ultrasound microscope as a function of intensity and pulse sequence. For example, the ultrasound transducer and microscope objective were focused to the same spatial location, and the image sequences were captured with a high-speed camera in either fluorescence or brightfield mode. The transducer can be fed with arbitrary waveforms and triggered from a LabVIEW program. Focused disruption of the described acoustically responsive vehicles (e.g., with the release of aqueous content such as payloads) was demonstrated in exemplary experiments. For example, nanoemulsions from liquid PFCs were prepared by using a probe-tip sonicator at high intensity on ice with fluorocarbons of various molecular weights, e.g., from perfluoropentane (C₅F₁₂) to perfluorononane (C₉F₂₀). A surfactant was used to stabilize the emulsions, allowing consistent generation of monodisperse emulsions with diameters around 150 nm. Using the described method, the emulsions were loaded into the liposomes. These nanoemulsions can create a site for a cavitation shockwave upon ultrasound exposure.

FIG. 7 shows an exemplary image sequence of the destruction of liposomes by focused ultrasound in real time. The exemplary image sequence features liposomes containing 150 nm liquid perfluorooctane nanoparticles (upper row) and control liposomes without nanoparticles (lower row) before, during and after exposure to focused ultrasound. Images 701 and 704 show the liposomes with and without nanoparticles, respectively, before focused ultrasound. Upon exposure to the focused ultrasound (2.25 MHz), the fluorescently labeled membranes were ruptured and liposomes totally destroyed as shown in images 702 and 703, and the effect had little to no dependence on the dispersant's boiling point. In the control liposomal samples with no nanoparticles, there was never any liposome destruction (as shown in images 705 and 706) at the same settings as nanoparticle-loaded liposomes. The liposome membrane is labeled with DiO. The free emulsion nanoparticles were removed by dialysis using a 0.4 μm polycarbonate membrane. As seen in the images, only liposomes with nanoparticles were destroyed. Size bar shown represents 2 μm.

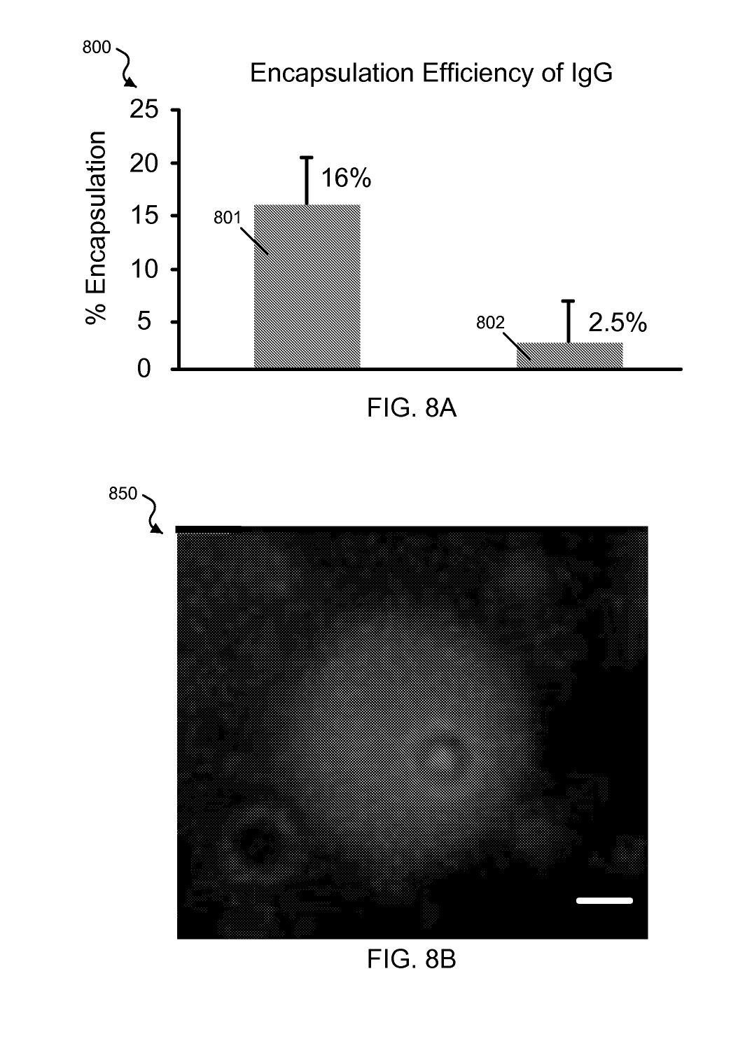

The disclosed technology has been implemented in several exemplary demonstrations that show several types of molecules successfully incorporated inside the engineered liposomes, e.g., drugs, proteins, enzymes, DNA inside liposomes, etc. The disclosed method can allow incorporation of a variety of molecules of different size and water solubility. For example, immunoglobulin IgG was dissolved in the aqueous medium during the liposome preparation as shown in FIG. 8A. FIG. 8A shows an exemplary plot 800 of encapsulation efficiency of IgG payload loading using the disclosed liposome fabrication method (bar 801) and a control method (bar 802). The loading of IgG was quantified using an enzyme-linked immunosorbent assay (ELISA). Control liposomes were prepared by standard dehydration-rehydration-vortexing methods, which resulted in a 7-fold lower encapsulation efficiency of 2.5% (bar 802). The exemplary method of the disclosed technology resulted in 16% efficiency of immunoglobulin incorporation (bar 801), suggesting that a large fraction of the volume is trapped upon the closure of lipid lamellar sheets. Similar to the liposome featured in data presented in FIG. 8A, chemotherapy agent doxorubicin has also been successfully incorporated into the liposome as shown in image 850 in FIG. 8B. Other payloads can also be incorporated in a similar manner.

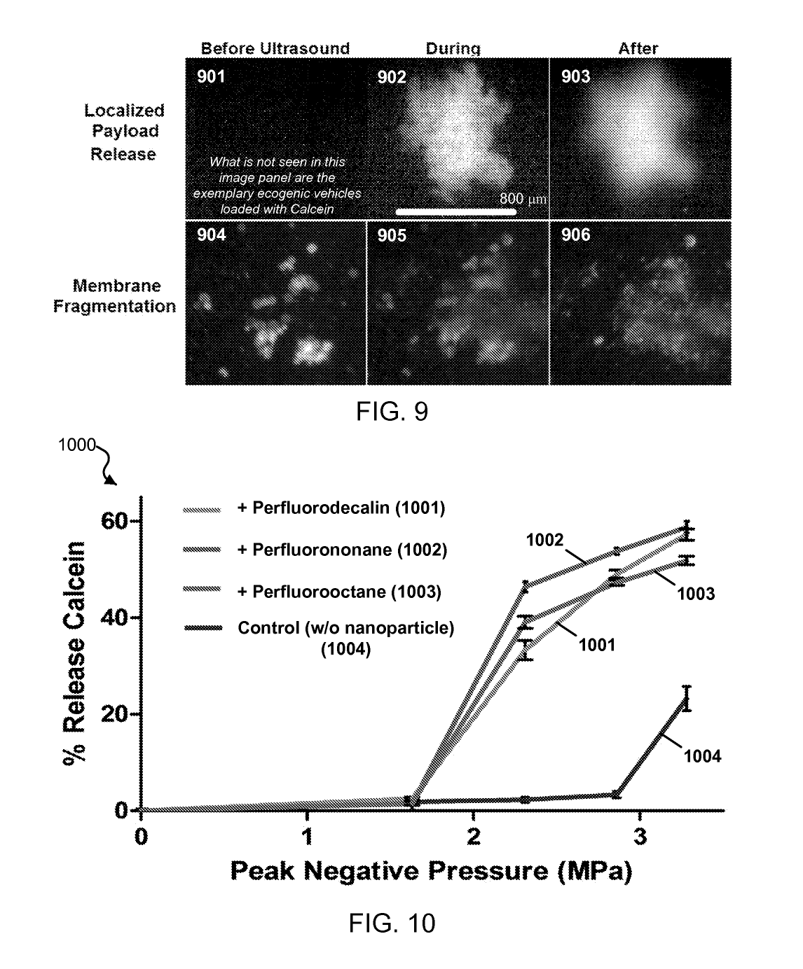

FIG. 9 shows other exemplary real-time observation images of liposome destruction. In this example, the top sequence shows the localized release of a calcein payload from exemplary nano-sized liposomes. The scale bar represents about 0.8 mm, which is approximately the size of the ultrasound focal region. Image 901 shows no fluorescence of the encapsulated calcein dye payload prior to ultrasound; image 902 shows exposure of calcein as determined by its observed fluorescence during ultrasound; and image 903 shows continued exposure of calcein after ultrasound. The bottom sequence shows an experiment that used larger liposomes whose membranes had been dyed with lipophilic dye DiO for visualization. Images 904 shows the liposomes intact before ultrasound; image 905 shows a frame during which the ultrasound occurs and liposomes are fragmented; and image 906 shows relatively complete fragmentation of the control liposomes after ultrasound. The bottom sequence demonstrates a confirmation of fragmentation of the lipid membrane, verifying the resulting payload release shown in images 902 and 903.

FIG. 10 shows an exemplary plot 1000 representing quantification of % release of calcein. Exemplary engineered liposomes were prepared using reverse phase evaporation to encapsulate an aqueous solution containing a 10 vol %˜100 nm perfluorocarbon emulsion and 23.75 mM calcein. Three different PFC emulsions were used, including perfluorodecalin (data line 1001), perfluorononane (data line 1002) and perfluorooctane (data line 1003). Control data is represented in plot 1000 as data line 1004. Dialysis with a large pore membrane removed the unencapsulated calcein and emulsions. Calcein released from ruptured liposomes resulted in dequenching of the dye and fluorescence increase, which allowed the quantification of % release.

Many types of nanoparticles that were tested and shown to nucleate cavitation and reduce the cavitation threshold. For example, various liquid PFC emulsions were implemented and some PFC emulsions were stabilized with two different types of surfactants. Solid nanoparticles of latex, polystyrene, gold, iron oxide and silver were implemented for comparison. These exemplary experimental implementations included loading liposomes with nanoparticles emulsions and insonating them with 2.25 MHz ultrasound. The ultrasound intensity required for liposome destruction was measured using an ultrasound microscope setup. Each of the exemplary liposome/nanoparticle samples are shown in Table 1. As seen in the table, threshold intensity was determined, from which below no disruption of the liposomes was observed. The threshold was seen to be dependent on the nanoparticle size and composition. The control liposomes without nanoparticles showed threshold intensity which was at least 40% higher.

TABLE 1
Ultrasound intensity to destroy nanoparticle-loaded liposomes

| Nanoparticle | Threshold Intensity (MPa) |
|---|---|
| PFHept-FSO-100 emulsion | ≦1.5 |
| PFO-FSO-100 emulsion | 1.24 |
| PFO-FSN-100 emulsion | 1.39 |
| PFN-FSN-100 emulsion | 1.39 |
| PFOB-FSN-100 emulsion | 1.39 |
| 100 nm dextran iron oxide | 1.5 |
| 100 nm Latex | 1.5 |
| 130 nm Latex | 1.16 |
| 500 nm Latex | No Response |
| 100 nm Ag | 1.5 |
| 200 nm Polystyrene | 1.16 |
| 500 nm Polystyrene | <0.62 |
| 100 nm Au | 1.5 |

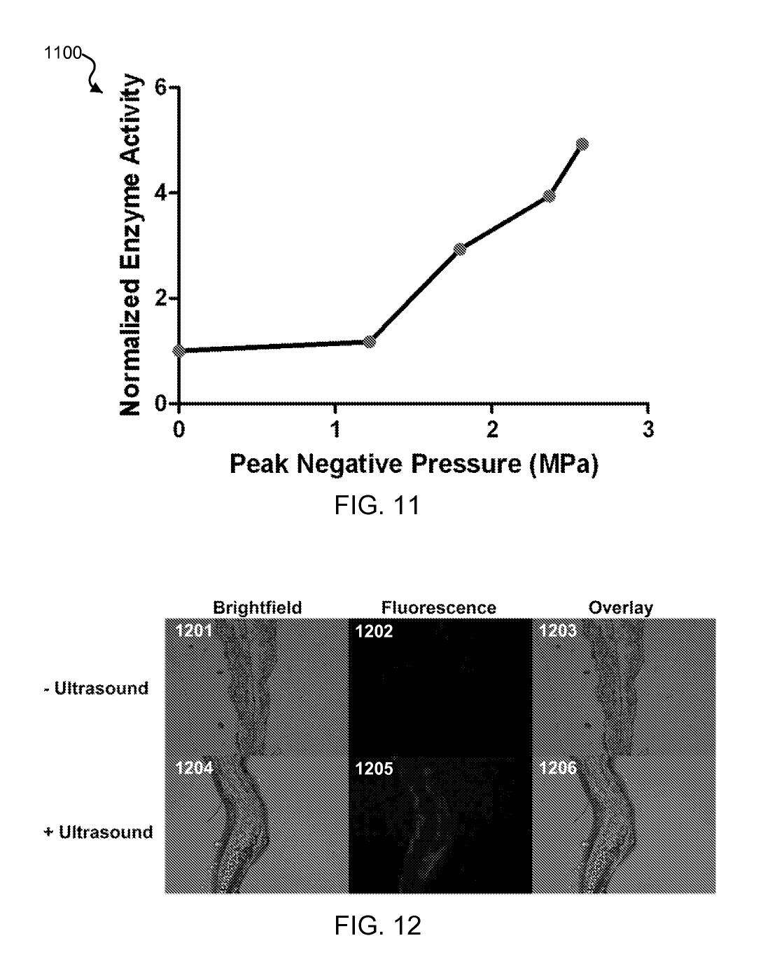

FIG. 11 shows an exemplary plot 1100 of normalized enzyme activity. In this example, liposomes were prepared containing the enzyme, beta-lactamase. Enzyme was released after the application of ultrasound of various pressures. For each pressure, the enzyme activity was measured by monitoring the absorbance increase using the chromogenic substrate, nitrocefin. The data shown in plot 1100 exemplifies the effects of increased peak negative pressure on increased enzyme activity.

FIG. 12 shows exemplary images of localized delivery of the disclosed technology in vivo, e.g., an in vivo demonstration of localized delivery to the ear of a mouse. Liposomes containing cavitation nucleation sites were injected into the tail vein of a mouse. Shown in FIG. 12 are two sections from the two different ears of the same mouse. One ear was insonated with ultrasound, which resulted in the local deposition of lipid membrane fluorescently labeled with lipophilic fluorophore DiR, as shown in images 1204, 1205, and 1206. Image 1204 shows the brightfield image; image 1205 shows the fluorescence image; and image 1206 shows the overlay image. The contralateral ear served as a “no ultrasound” control. As seen in images 1201, 1202, and 1203, there was no significant deposition of fluorescent membrane in this ear.

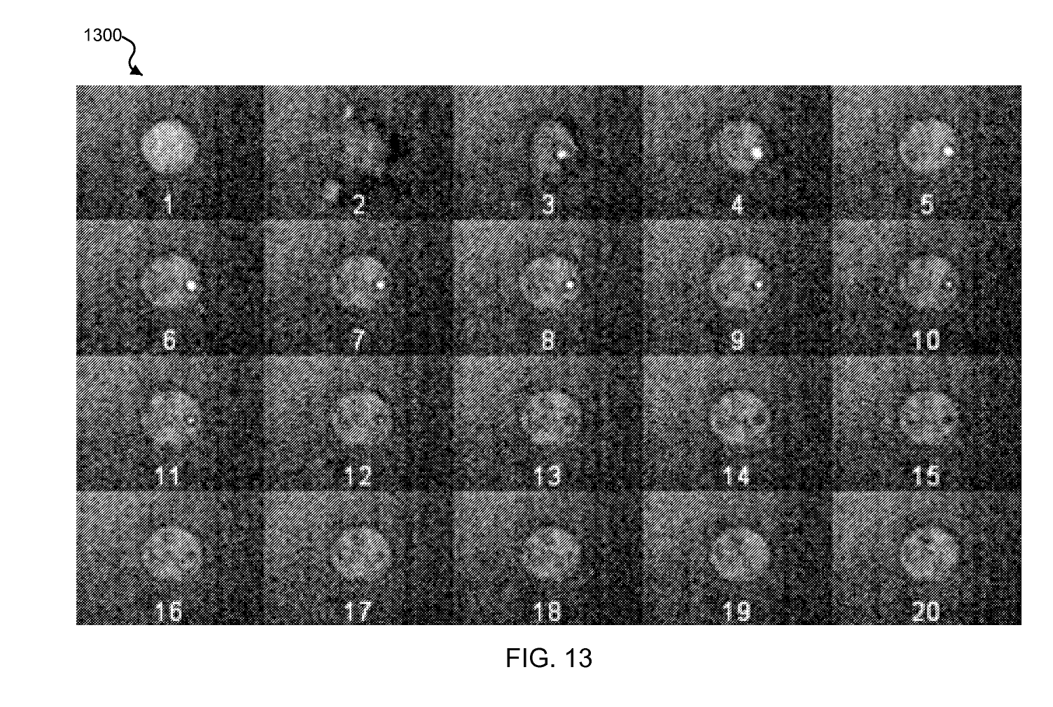

One exemplary method of nucleation of cavitation with emulsions can include transient formation of a vapor bubble at the surface or within the volume of the emulsion. For example, perfluorocarbon emulsions are able to produce such effects when insonified with ultrasound. Newly formed bubbles can condense or dissolve back into the emulsion or surrounding medium, rendering the effect repeatable for many applications of ultrasound (e.g., as shown in FIG. 13). Liquids such as perfluorocarbons used in the emulsion can be saturated with a high concentration of gasses, like oxygen or carbon dioxide, which can result in a reduction in the cavitation threshold.

FIG. 13 shows an exemplary series of high speed observation images 1300 of a perfluorocarbon emulsion nucleating acoustic cavitation. Frame 1 shows the emulsion before application of ultrasound. An ultrasound pulse occurs during frame 2, which shows a violent cavitation response, resulting in the production of a gas bubble seen in frame 3. Subsequent frames show the condensation or dissolution of the vapor bubble into the emulsion droplet until the bubble is no longer visible, and the emulsion again appears as it did in frame 1. Because the emulsion reverts to its original state, this process was repeatable over many ultrasound pulses.

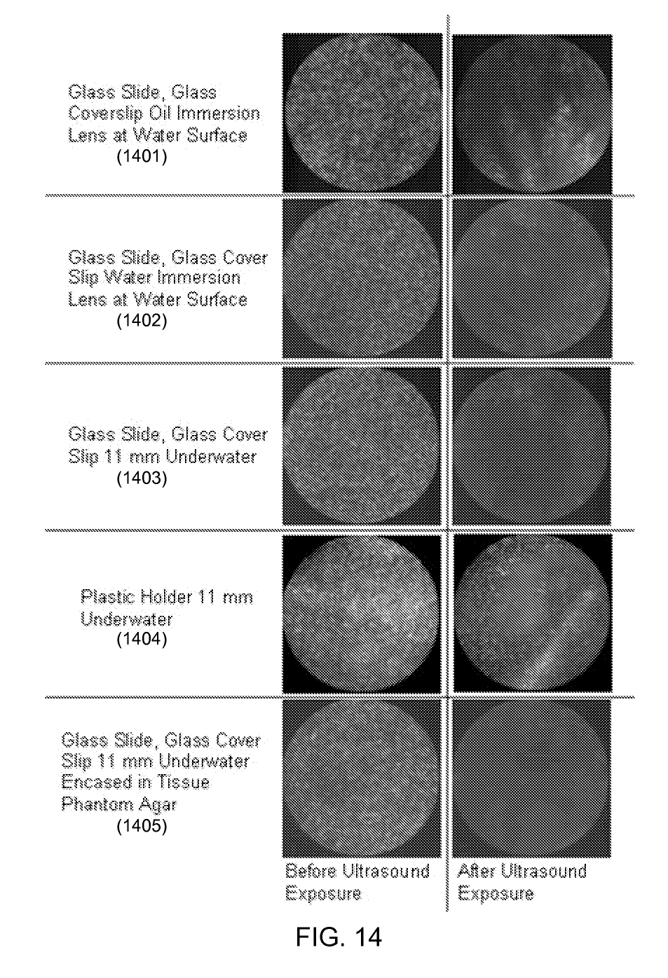

FIG. 14 shows exemplary images from a series of experiments confirming that the cavitation threshold reduction phenomena observed with iron oxide nanoparticles was not just an artifact of the experimental setup. The experimental configuration was changed using different arrangements before and after focused ultrasound, including condition 1401 of a glass slide, glass cover slip, and oil immersion lens at the water surface; condition 1402 of a glass slide, glass cover slip, and water immersion lens at the water surface; condition 1403 of a glass slide, glass cover slip, and 11 mm underwater; condition 1404 of a plastic holder and 11 mm underwater; and condition 1405 of a glass slide, glass cover slip, and 11 mm underwater encased in tissue phantom agar. For example, the holder was changed from being a glass cover slip on a glass slide to two pieces of thin plastic sheet held together in a plastic framework. The lens was changed from being an oil immersion type to a water immersion type. The location of the holder was changed from being at the air water interface to 11 mm underwater, and also an additional condition had the holder encased in agar tissue phantom to show that the cavitation threshold reduction can also occur in real tissue. This exemplary experimental implementation can provide confirmation that the observed effects are not an artifact of the type of lens used, the glass, or being at the air/water interface. As can be seen in the exemplary conditions of FIG. 14, the iron oxide nanoparticles cause cavitation to occur, which ruptures the surrounding fluorescent liposomes creating a debris field of lipid particles.

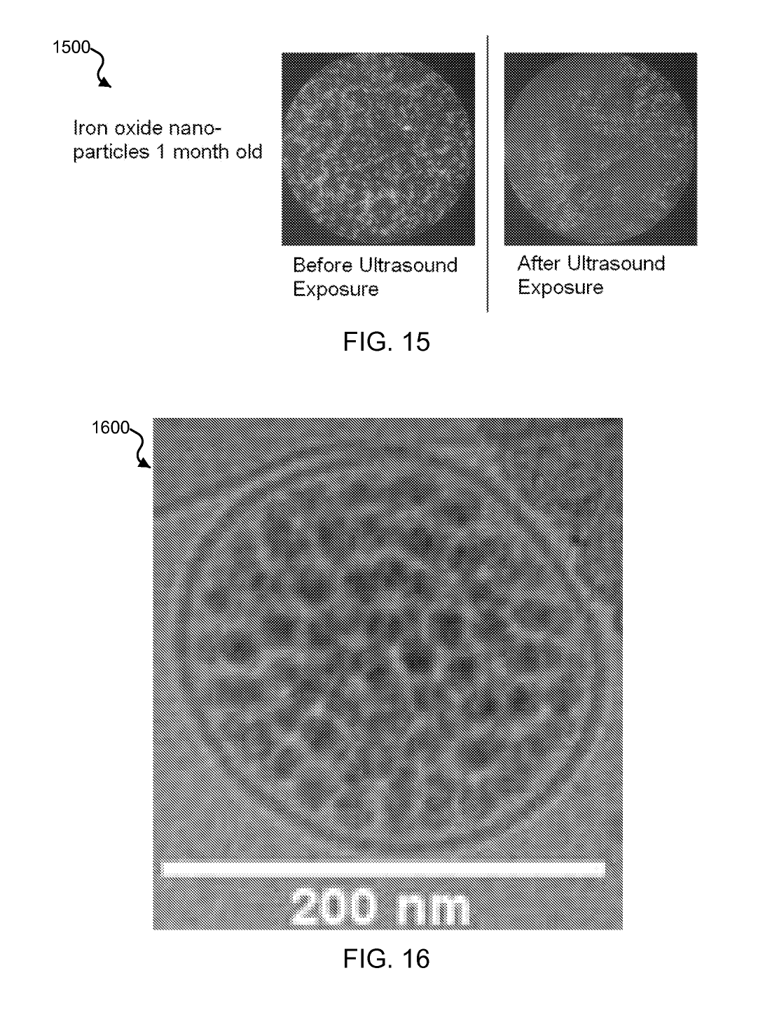

FIG. 15 shows an exemplary image 1500 of iron oxide nanoparticles after a month since fabrication. Image 1500 can provide confirmation that the iron oxide nanoparticles have not lost their cavitation threshold reduction properties after 1 month of being stored on the shelf.

FIG. 16 shows an exemplary cryogenic transmission electron micrograph (cryo-TEM) 1600 of a liposome containing densely-packed perfluorononane emulsions. Exemplary methods of the disclosed technology described previously were implemented to obtain this experimental image. For example, one drop of the liposome solution was placed on a copper grid and subsequently vitrified in liquid ethane, preserving the liposome structure and internalized state of the emulsions. Tomograms were generated to verify that the emulsions were in fact encapsulated in the liposome's volume, rather than on the surface.

The subject matter described in this patent document can be implemented in specific ways that provide one or more of the following features and advantages. The disclosed technology can be used in variety of exemplary applications, including: rupturing the delivery vehicle for releasing its payload; producing stabilized gas microbubbles from encapsulated nanodroplets for contrast enhanced ultrasound imaging; producing UV-blue light to perform local photochemistry to turn a prodrug in to its therapeutic form; employing the disclosed acoustically responsive vehicles with high intensity focused ultrasound (HIFU) to enhance ultrasound imaging concurrent to producing localized heat to alter materials properties; producing characteristic sound emission for guided therapy applications. For example, the described acoustically responsive vehicles can reduce the cavitation threshold to ultrasound levels below that of levels that may otherwise cause cavitation in the absence of any vehicles, and potentially cause harm to a patient's body. For example, the described acoustically responsive vehicles can transport a variety of payloads. Exemplary payloads can include one or more of the following: a hydrophilic drug, e.g., which can be dissolved in the aqueous space; a hydrophobic drug, e.g., which can be contained in a nanoparticle or a micellar structure; a nanoparticle, e.g., which can be or contain imaging or therapeutic agents including iron oxide, gadolinium, or other materials; a gas bubble or fluorocarbon droplets or emulsion as contrast agents for ultrasound; a radioactive isotope; an encapsulated enzyme; an encapsulated virus; and an encapsulated nucleic acid, among others.

Implementations of the disclosed technology can be applied in a variety of biomedical fields including targeted drug delivery. For example, applications can include delivery of chemotherapy drugs to tumors and delivery of therapeutic drugs specifically to desired (non-cancerous) tissues within the body. Other exemplary applications can include the local delivery of enzymes for the digestion of interstitial tissues in tumors and for other substrate conversion. Local and controlled delivery of nucleic acids can be implemented, for example, for gene delivery or gene silencing applications. Also, the disclosed technology can include producing stabilized microbubbles for enhanced ultrasound imaging. The disclosed technology can be used to concurrently deliver payloads to a targeted site (e.g., delivery of drugs, enzymes, prodrugs, genetic materials, etc. to a target cell or tissue) and enhance ultrasound imaging resolution of the targeted site. The disclosed fabrication methods can be used to create cell like compartments for the containment of biological machinery and/or imaging agents.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described above should not be understood as requiring such separation in all embodiments.

Only a few implementations and examples are described and other implementations, enhancements and variations can be made based on what is described and illustrated in this patent document.

1. An ultrasound system for delivering a substance loaded in an acoustically responsive particle, comprising:
a mechanism that supplies one or more particles each having an outer shell that encloses an aqueous medium containing an acoustic-sensitizer particle and a payload substance; and
a mechanism that produces ultrasonic acoustic energy and directs the ultrasonic acoustic energy at a particular region where the one or more particles are located to cause cavitation within the acoustic-sensitizer particle or in the aqueous medium at an interface with the acoustic-sensitizer particle that ruptures the outer shell of the one or more particles, thereby releasing the payload substance within the particular region.

2. The ultrasound system of claim 1, wherein the one or more particles further include molecules attached to the outer shell, the molecules including at least one of polyethylene glycol, ligands, imaging agents, drugs, enzymes, nucleic acids, or other bimolecular agents.

3. (canceled)

4. The ultrasound system of claim 1, wherein the payload substance includes at least one of a drug, an imaging agent, an enzyme, a prodrug, a nucleic acid, a viral vector, a therapeutic or sensing substance, or a component that aids in internalization of another payload substance by a cell.

5. The ultrasound system of claim 1, wherein the acoustic-sensitizer particle includes at least one of a nano-sized liquid emulsion droplet, a metal nanoparticle, an oxide nanoparticle, a polymer nanoparticle, a biomolecule, or a solid or liquid particle stabilizing one or more nano-scale pockets of a gas.

6. The ultrasound system of claim 1, wherein the one or more particles include at least one of a liposome, a polymersome, an inorganic or organic shell or capsid, or a biological cell.

7. The ultrasound system of claim 1, wherein the mechanism that supplies one or more particles is configured to supply the one or more particles into a living body and the particular region includes a tumor cell, a stem cell, a white blood cell, or a cell in an organ.

8. The ultrasound system of claim 7, wherein the one or more particles are resilient to damage in the living body outside of the particular region, including damage caused by pressure, pH, temperature, or chemical substances in the living body.

9. The ultrasound system of claim 7, wherein the one or more particles have a cavitation threshold at an ultrasound intensity and frequency level below that of levels that cause harm to the living body.

10. A drug delivery vehicle, comprising:
a carrier comprising an outer membrane having an interior including an aqueous medium and an acoustic sensitizer particle and a payload substance within the aqueous medium, the carrier capable of being delivered to a target tissue in a body,
wherein the acoustic sensitizer particle is structured to reduce an acoustic cavitation threshold through cavitation within the acoustic-sensitizer particle or at an interface between the acoustic sensitizer particle and the aqueous medium.

11. The drug delivery vehicle of claim 10, wherein the carrier includes at least one of a liposome, a polymersome, an inorganic or organic shell or capsid, or a biological cell.

12. The drug delivery vehicle of claim 10, wherein the acoustic sensitizer particle includes a particle in a solid form, a liquid form, or a liquid or solid form stabilizing one or more nano-scale pockets of a gas.

13. The drug delivery vehicle of claim 10, wherein the target tissue includes a biological tissue including a tumor cell, a stem cell, a white blood cell or a cell in an organ.

14. The drug delivery vehicle of claim 41, wherein the agent to increase circulation time includes at least one of a polyethylene glycol, a zwitterionic compound, or a patient-specific coating such as cell membranes.

15. The drug delivery vehicle of claim 10, wherein the drug delivery vehicle is made of a bioresorbable material.

16. (canceled)

17. (canceled)

18. (canceled)

19. A method for producing a stabilized microbubble, comprising:
applying ultrasound energy to a liposome containing a nanoemulsion structure in an aqueous medium to fragment the liposome, causing lipids of the fragmented liposome to rearrange into one or more microbubbles,
wherein the lipids that form the one or more microbubbles stabilize the one or more microbubbles against coalescence or dissolution.

20. (canceled)

21. (canceled)

22. (canceled)

23. (canceled)

24. (canceled)

25. An ultrasound contrast agent device, comprising:
an outer membrane structured to form an enclosed chamber, wherein the outer membrane is at least one of a liposome, polymersome, an inorganic or organic shell or capsid, or a biological cell;
an aqueous medium enclosed within the chamber;
a payload substance in the aqueous medium within the chamber, wherein the outer membrane protects the payload substance from degradation or opsonization; and
one or more nanoemulsion structures in the aqueous medium within the chamber,
wherein the outer membrane is formed of a material that can be fragmented by ultrasound energy into components that rearrange into one or more microbubbles that are stabilized against coalescence or dissolution and operate as a contrast agent in ultrasound imaging, and
wherein the outer membrane is functionalized with at least one of a targeting ligand to cause the device to selectively accumulate in a particular region or an agent to increase circulation time by reducing uptake from undesired body tissues, organs, and systems.

26. (canceled)

27. (canceled)

28. (canceled)

29. (canceled)

30. (canceled)

31. The ultrasound contrast agent device of claim 19, wherein the ultrasound energy directed at the outer membrane releases the payload substance.

32. (canceled)

33. An ultrasound responsive device for carrying a payload substance, comprising:
an outer membrane structured to form an enclosed chamber;
an aqueous medium enclosed within the chamber;
one or more ultrasound-responsive nanoparticles in the aqueous medium and structured to reduce an ultrasound cavitation threshold at an interface between each ultrasound-responsive nanoparticle and aqueous medium; and
a payload substance in the aqueous medium,
wherein the outer membrane is formed of a material that can be fragmented by cavitation within the chamber in response to ultrasound energy to release the payload substance outside the enclosed chamber.

34. (canceled)

35. The ultrasound responsive device of claim 33, wherein the outer membrane is at least one of a liposome, polymersome, an inorganic or organic shell or capsid, or a biological cell.

36. The ultrasound responsive device of claim 33, wherein the payload substance includes at least one of a drug, an imaging agent, an enzyme, a prodrug, a nucleic acid, a viral vector, a therapeutic or sensing substance, or a component that aids in internalization of another payload substance by a cell.

37. The ultrasound responsive device of claim 33, wherein the one or more ultrasound-responsive nanoparticles include at least one of a nano-sized liquid emulsion, a metal, an oxide, a polymer, a biomolecule, or a particle stabilizing nano-scale pockets of a gas.

38. The drug delivery vehicle of claim 10, wherein the payload substance includes at least one of a drug, an imaging agent, an enzyme, a prodrug, a nucleic acid, a viral vector, a therapeutic or sensing substance, or a component that aids in internalization of another payload substance by a cell.

39. The drug delivery vehicle of claim 10, wherein the outer membrane protects the acoustic sensitizer particle and the payload substance from degradation or opsonization.

40. The drug delivery vehicle of claim 10, wherein an outer surface of the outer membrane is functionalized with a targeting ligand to cause the drug delivery vehicle to selectively accumulate in a target tissue region over other tissues.

41. The drug delivery vehicle of claim 40, wherein the outer surface of the outer membrane is further functionalized with an agent to increase circulation time by reducing uptake from undesired body tissues, organs, and systems.

42. The method of claim 19, further comprising:
functionalizing the liposome with at least one of a targeting ligand to cause the selectively accumulation in a target region or an agent to increase circulation time by reducing uptake from undesired regions outside of the target region.
